(19) United States
(12) Patent Application Publication
(10) Pub. No.: US 2025/0204986 A1
(43) Pub. Date: Jun. 26, 2025

(54) END EFFECTOR ASSEMBLIES FOR SURGICAL INSTRUMENTS

(71) Applicant: The University of North Carolina at Charlotte, Charlotte, NC (US)

(72) Inventors: Nathaniel Michael Fried, Concord, NC (US); Patrick J. O’Brien, Weddington, NC (US); Woheeb Muhammad Saeed, Horsham, PA (US)

(73) Assignee: The University of North Carolina at Charlotte, Charlotte, NC (US)

(21) Appl. No.: 19/075,384

(22) Filed: Mar. 10, 2025

Related U.S. Application Data
(63) Continuation-in-part of application No. PCT/US2023/032379, filed on Sep. 11, 2023.

(51) Int. Cl. A61B 18/22; A61B 17/00; A61B 17/29; A61B 18/00

(52) U.S. Cl. CPC A61B 18/22; A61B 2017/00057; A61B 2017/00907; A61B 17/2909; A61B 2017/2926; A61B 2018/00184; A61B 2018/00601; A61B 2018/0063; A61B 2018/00642; A61B 2018/2272

(57) ABSTRACT

Surgical instruments, end effectors for surgical instruments, and methods of using surgical instruments. An example end effector assembly includes a first jaw member and a second jaw member. The first jaw member includes: a transparent tissue contacting surface; at least one transparent viewing portion; a fluid-tight cavity configured to receive an optical fiber; and a reflector configured to reflect a substantial portion of light from the optical fiber towards the transparent tissue contacting surface. The second jaw member is configured to move towards the transparent tissue contacting surface of the first jaw member.

### PRIORITY CLAIM

This application is a continuation in part of PCT International Patent Application Serial No. PCT/US2023/032379, filed Sep. 9, 2023, which claims the priority benefit of U.S. Provisional Patent Application Ser. No. 63/404,980, filed Sep. 9, 2022, the disclosure of each of which is incorporated herein by reference in its entirety.

### GOVERNMENT INTEREST

This invention was made with government support under R15 EB028576 awarded by the National Institutes of Health (NIH). The government has certain rights in the invention.

### TECHNICAL FIELD

The subject matter disclosed herein relates generally to end effector assemblies for surgical instruments and methods for using the end effector assemblies and surgical instruments.

### BACKGROUND

Energy-based, ultrasonic (US) and radiofrequency (RF) devices commonly provide rapid sealing and hemostasis of vascular tissues during surgery. These devices are used in about 80% of the 15 million laparoscopic surgical procedures performed globally each year. As an alternative, infrared (IR) laser sealing and bisection of vascular tissues has recently been reported in the laboratory. Potential advantages of IR laser devices include: (1) rapid optical sealing and cutting of vascular tissues without the need for a separate deployable mechanical blade to bisect tissue seals, (2) less thermal spread for potential use near sensitive tissue structures (e.g. nerves), (3) stronger vessel seals as measured by burst pressures (BP) in the laboratory, and (4) lower device jaw temperatures for a safer profile (e.g. to avoid thermal damage to adjacent soft tissues through inadvertent device-tissue contact) and to enable shorter device cooling times in between successive applications for reduced operating room times with associated cost savings.

A major design limitation is the size constraints of the standard Maryland style jaw and the 5-mm-outer-diameter (OD) shaft of laparoscopic energy-based surgical devices. The bottom fixed jaw design will not only need to reflect the IR laser beam at a 90° angle, but also to convert the circular spatial beam profile into a linear beam, to create a uniform lengthwise seal across the width of the blood vessel, all within the limited space of the laparoscopic instrument. In general, the top jaw, with a pivoting hinge, serves to open and close for grasping vessels and provides tissue compression to facilitate thermal sealing.

Another design limitation is the metallic materials used for jaws in standard laparoscopic devices. The limited surgical field-of-view provided by these non-transparent materials may make accurate positioning and centering of tissues within the jaws more difficult to achieve in clinical practice.

### SUMMARY

This document describes surgical instruments, end effectors for surgical instruments, and methods of using surgical instruments. An example end effector assembly includes a first jaw member and a second jaw member. The first jaw member includes: a transparent tissue contacting surface; at least one transparent viewing portion; a fluid-tight cavity to keep bodily fluids out, configured to receive an optical fiber; and a reflector configured to reflect a substantial portion of light from the optical fiber towards the transparent tissue contacting surface. The second jaw member is configured to move towards the transparent tissue contacting surface of the first jaw member.

Although some of the aspects of the subject matter disclosed herein have been stated hereinabove, and which are achieved in whole or in part by the presently disclosed subject matter, other aspects will become evident as the description proceeds when taken in connection with the accompanying drawings as best described hereinbelow.

### BRIEF DESCRIPTION OF DRAWINGS

FIG. 1A shows an example surgical instrument;

FIG. 1B illustrates the end effector assembly;

FIG. 1C illustrates that the second jaw member is configured to move towards the transparent tissue contacting surface of the first jaw member;

FIG. 1D is a cross-sectional view of a first example configuration of the first jaw member;

FIG. 1E is a cross-sectional view of a second example configuration of the first jaw member;

FIG. 1F is a flow diagram of an example method for using the surgical instrument.

FIGS. 2A-2B illustrate an example system for testing an example implementation of the end effector;

FIG. 3 is a graph of irradiance versus spatial position (mm) that illustrates the initial laser beam profile exiting the optical fiber;

FIG. 4 shows the spatial distribution of the laser beam exiting the optical chamber for quartz and sapphire;

FIG. 5 illustrates Monte Carlo simulations showing light transport through the quartz chamber and into the tissue layer;

FIG. 6 shows images of the quartz chamber design and thermal simulation results at several time points;

FIG. 7 shows simulated results for the temperature-time response on the external surface of the quartz chamber;

FIGS. 8A-8B show representative temperature-time data for TCs placed on the external and internal surfaces of the quartz and sapphire chambers;

FIG. 9 shows a scatter plot of BPs as a function of vessel diameter for quartz and sapphire chambers; and

FIG. 10 shows representative blood vessels after laser treatment, for quartz and sapphire chambers.

FIG. 11 illustrates an example system for testing an example implementation of the end effector similar to the example system described in FIGS. 2A and 2B.

FIG. 12 illustrates an example system for acquiring the optical transmission signal during continuous-wave laser ablation of compressed porcine renal blood vessels. (Left top inset) Start of carbon layer formation on the tissue surface during laser irradiation. (Right top inset) Formation of an ablation crater during laser irradiation.

FIG. 13A is a photograph of a vessel after bisection using 59 W for 5 s. A fascia layer is visible and the vessel was easily detached by pulling uniformly from both sides. (d=3.2 mm, BP1: 460 mmHg, BP2: 776 mmHg).

FIG. 13B is a photograph of a vessel after bisection using 59 W for 6 s. A clean cut is shown (d=3.5 mm, BP1: 1160 mmHg, BP2: 372 mmHg).

FIG. 13C shows a comparison of laser incident power and irradiation times for each data set. Laser irradiation times shorter than 5 s resulted in tissue cutting, but not sealing, presumably due to insufficient thermal conduction and thermal spread during the laser treatment time. Laser incident powers less than 41 W failed to produce a full-thickness cut, due to insufficient irradiance.

FIG. 14 shows optical transmission signal as a function of time during CW laser tissue ablation and (inset) the representative tissue sample (d=3.2 mm).

FIG. 15 illustrates an example laparoscopic device with quartz jaws and optical feedback system. The therapeutic, reciprocating, side-firing, optical fiber is housed in the bottom fixed jaw, while a linear array of four photodiodes and a neutral density filter for diagnostic feedback, is housed in the upper, hinged, active jaw.

FIG. 16 illustrates an example system for optical beam characterization.

### DETAILED DESCRIPTION

There are several technical limitations of systems using a reciprocating, side-firing, optical fiber to produce a uniform linear beam profile within a standard Maryland laparoscopic jaw design. First, the non-transparent, metallic jaws of standard laparoscopic devices and the corresponding limited surgical field-of-view may make accurate positioning and centering of tissues within the jaws difficult to achieve in clinical practice.

Second, a wide range of blood vessel diameters (2-6 mm), are typically treated during surgery with energy-based devices. Therefore, a fixed scan length for the reciprocating fiber is not practical. A short scan length may not provide a full-thickness seal in larger vessels, leading to incomplete and failed seals without hemostatic closure. A long scan length may result in inefficient deposition of the optical energy into the vessel and excessive laser energy being transmitted around the edges of a small vessel, in turn resulting in higher device jaw temperatures and longer cooling times in between applications than acceptable during surgery.

Disclosed herein are end effector assemblies for surgical instruments. The end effector assemblies have a transparent viewing portion that may enable improved visibility for positioning vascular tissues within the laparoscopic device jaws and customization of the reciprocating fiber scan length to match the compressed width of the blood vessels.

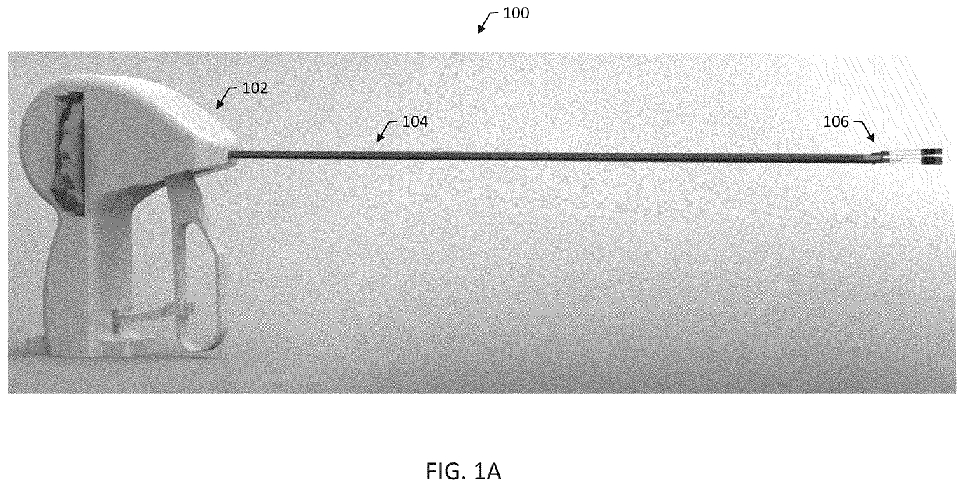

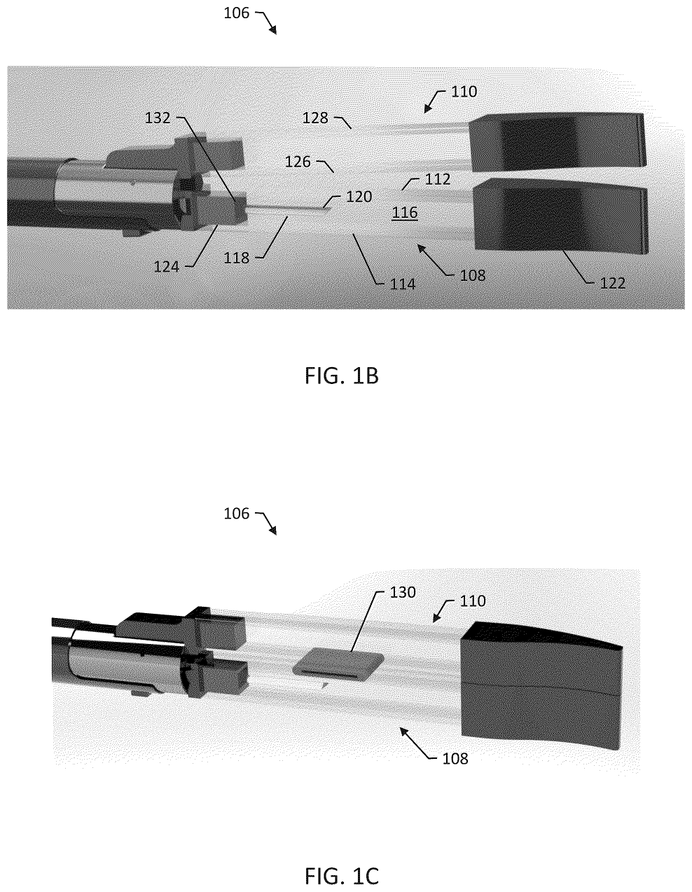

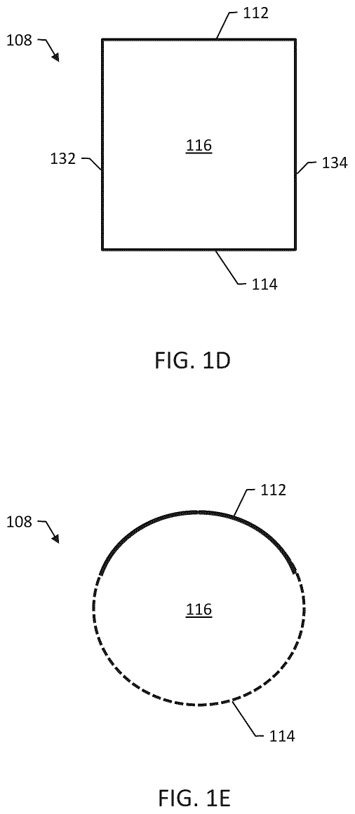

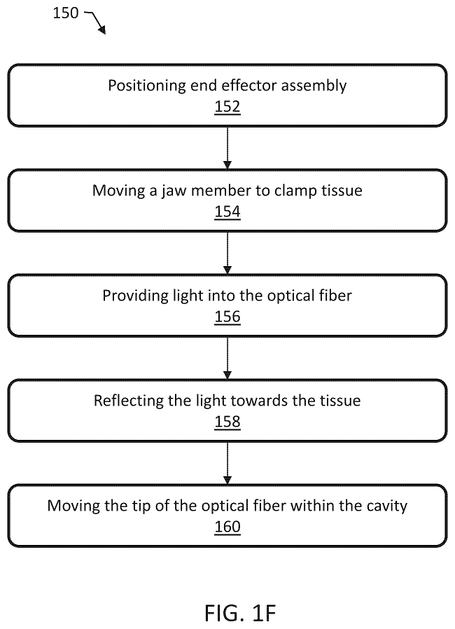

FIG. 1A shows an example surgical instrument 100. The surgical instrument includes a handle 102, an elongated body 104 extending from the handle 102, and an end effector assembly 106 secured to a distal portion of the elongated body 104. The handle 102 includes one or more control interfaces configured to manipulate the end effector assembly 106. The control interfaces can include, for example, a movable handle, a trigger, a switch, and a button. The handle 102 can include a wheel or rotation control configured to rotate the elongated body 104, and the end effector assembly 106, relative to the handle 102.

FIG. 1B illustrates the end effector assembly 106 in detail. The end effector assembly 106 comprises a first jaw member 108 and a second jaw member 110. The first jaw member 108 includes a transparent tissue contacting surface 112 and at least one transparent tissue viewing portion 114. The first jaw member 108 includes a fluid-tight cavity 116 to keep fluid out and configured to receive an optical fiber 118. In some examples, the fluid-tight cavity 116 is sufficiently sealed to be air-tight.

The transparent tissue contacting surface 112 and the transparent tissue viewing portion 114 can be made, for example, from quartz, sapphire, or any other appropriate material. The term “transparent” is used in this document to refer to material that substantially transmits light in the visible range of 400-700 nm for the surgeon to see through the device and in the infrared range at a wavelength suitable for sealing or cutting tissue or both. For example, the optical fiber 118 may transmit light having a wavelength in a range of about 800 nm to about 2500 nm.

The first jaw member 108 includes a reflector 120 configured to reflect a substantial portion of light from the optical fiber 118 towards the transparent tissue contacting surface 112. In some examples, the reflector 120 comprises a side-firing fiber tip of the optical fiber 118, e.g., created by an angled tip. In some examples, a small mirror or other optical element is positioned within the cavity 116 to direct light exiting the optical fiber 118 towards the tissue contacting surface 112.

The first jaw member 108 can include a first opaque plug 122 at a distal end of the first jaw member 108. The first jaw member 108 can include a second opaque plug 124 at a proximal end of the first jaw member 108. The opaque plugs 122 and 124 can be useful, for example, to prevent stray light from exiting the first jaw member 108, and to close the distal tips of the optical chambers to provide fluid-tight closure.

The first jaw member can include one or more mounts 132 configured to prevent the optical fiber 118 from rotating within the fluid-tight cavity 116. The mounts 132 can be useful, for example, where the reflector 120 is an angled fiber tip that should remain oriented towards the tissue contacting surface 112.

FIG. 1C illustrates that the second jaw member 110 is configured to move towards the transparent tissue contacting surface 112 of the first jaw member 108. The second jaw member 110 can include a transparent tissue contacting portion 126 opposing the transparent tissue contacting portion 112 of the first jaw member 108 and a transparent viewing portion 128. For example, the second jaw member 110 can be coupled with the first jaw member 108 by a hinge, allowing an operator to control the second jaw member 110 to move towards the first jaw member 108 to clamp tissue 130 between the first jaw member 108 and the second jaw member 110.

In some examples, the tissue contacting surface 112 of the first jaw member 108 or the tissue contacting surface 126 of the second jaw member 110 or both includes features to prevent tissue 130 grasped or clamped between the first and second jaw members 108 and 110 from moving relative to the first and second jaw members 108 and 110. For example, one or more of the tissue contacting surfaces may be textured to grasp tissue. Additionally or alternatively, one or more of the tissue contacting surfaces 112 and 126 may include ridges, ribs, or other features extending towards the opposite jaw member to secure tissue between the first and second jaw members.

FIG. 1D is a cross-sectional view of a first example configuration of the first jaw member 108. The first jaw member 108 comprises a substantially rectangular tube having four sides 112, 114, 132, and 134. The transparent tissue contacting surface 112 is a first side and the transparent viewing portion is a second side opposite the first side. The other sides 132 and 134 can also be transparent (or include transparent portions) to further allow for viewing through the first jaw member 108.

FIG. 1E is a cross-sectional view of a second example configuration of the first jaw member 108. The first jaw member 108 comprises a tube having a substantially circular cross-section. The entire tube may be transparent. A first arc segment of the tube is considered the transparent tissue contacting surface 112 and a second arc segment of the tube is considered the transparent viewing portion 114.

The surgical instrument 100 can be used for laparoscopic surgery. FIG. 1F is a flow diagram of an example method 150 for using the surgical instrument 100.

The method 150 includes positioning the end effector within a patient (152), e.g., using one or more control interfaces on the surgical instrument. The method 150 includes moving the second jaw member towards the first jaw member to clamp tissue between the first jaw member and the second jaw member (154).

The method 150 includes providing light into the optical fiber of the end effector assembly (156). Light can be provided from any appropriate light source, e.g., lasers, light emitting diodes (LEDs), and lamps. The method 150 includes reflecting a substantial portion of light from the optical fiber towards the transparent tissue contacting surface of the first jaw member (158), for example, using a side-firing optical fiber tip.

The method 150 includes moving the tip of the optical fiber within the fluid-tight cavity of the first jaw member and thereby cutting or sealing tissue or both (160). For example, a motor controller can be used to move the tip of the optical fiber in a reciprocating manner. The distance moved by the fiber optic tip (scan length) can be controlled based on the width of the compressed tissue clamped, e.g., such that the distance is greater for larger vessels. The size of a vessel can be judged more easily (e.g., by a physician) due to the transparent viewing portion of the end effector.

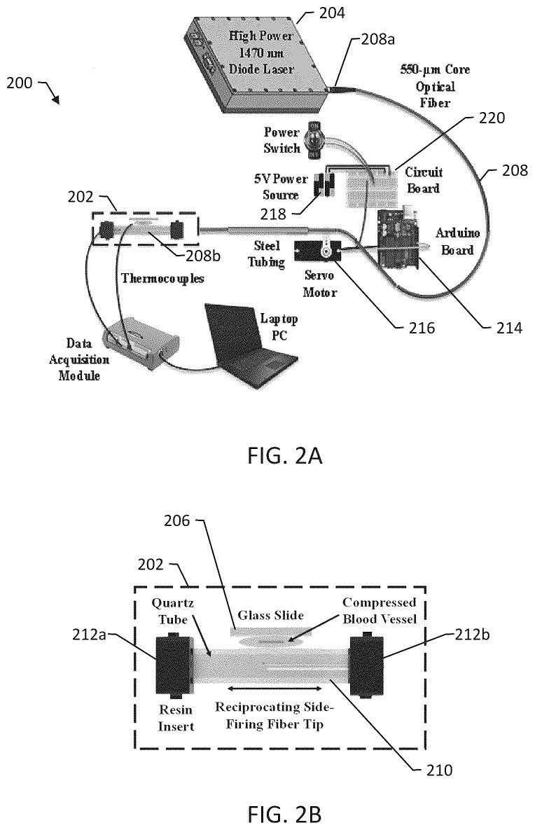

FIGS. 2A-2B illustrate an example system 200 for testing an example implementation of the end effector 202 using quartz and sapphire tubing. Results of the testing are presented below for the purpose of illustration and not limitation.

Optical and thermal characterization of the quartz and sapphire tubing was performed. Infrared laser sealing of porcine renal blood vessels was also conducted to determine whether industry standard destructive vessel burst pressure (BP) measurements in the laboratory are sufficient for potential future surgical application, as judged by BP needing to exceed both systolic (120 mmHg) and hypertensive blood pressure (180 mmHg).

Fresh porcine kidney pairs were acquired and renal arteries were dissected and then stored in physiological saline in a refrigerator prior to use. A similar range of vessel diameters was chosen for the quartz and sapphire treatment groups, for direct comparison. A total of n=13 blood vessels with a mean, uncompressed diameter of 3.4±0.7 mm, were selected for tests with the quartz chamber, while a total of n=14 vessels with a diameter of 3.2±0.7 mm, were selected for use with the sapphire chamber (P=0.41).

A 100-Watt, 1470 nm wavelength IR diode laser 204 was used for vessel sealing studies. The laser was operated in continuous-wave (CW) mode with incident power at the tissue surface of 30 W for a short duration of 5 s. Laser power output was calibrated using a meter and detector.

Blood vessel samples were compressed and fixed in place using a 0.5-mm-thick optical window 206 locked in a clamp, to simulate a transparent top jaw. An optical coherence tomography (OCT) system with 8 Fr (2.67-mm-OD) laparoscopic probe provided non-invasive imaging and measurement of the compressed vessel thickness to confirm consistent pressures and for reproducible measurements between samples. The compressed tissue thickness was fixed at 0.4 mm to approximately match the optical penetration depth of IR light in water-rich soft tissues at a wavelength of 1470 nm, and to provide uniform, full-thickness seals.

A low-OH, silica optical fiber 208 with 550-μm-core, 600-μm-cladding, 1040-μm-jacket, and numerical aperture (NA) of 0.22 was used for vessel sealing studies. The proximal fiber tip 208a with high-power, SMA905 connector was attached to the laser 204. The side-firing, distal fiber tip 208b was prepared using a bare fiber polisher, rotated to achieve a 50° angle, and accurate to 0.5°. A value of 90% power reflected was acceptable due to the accuracy of the side angle polish.

The bottom optical chamber 210 comprised quartz or sapphire square tubing with dimensions of 1.8×1.8 mm ID, 2.7×2.7 mm OD, 25 mm length, and 0.45 mm wall thickness. A 3D-printed, black resin plug 212a-b was placed on each end. The proximal end plug 212b had a small hole to allow insertion of the optical fiber 208. The distal end plug 212a also had a small hole to allow insertion of a thermocouple, but otherwise provided fluid-tight closure and absorbed stray light in the forward direction. The fiber 208 was inserted into the quartz/sapphire tubing 210 and clamped in place, leaving 0.6 mm between the side-firing silica fiber tip and the inner walls of the tubing. The distance from fiber tip to vessel wall was measured to be 1.05 mm (air gap of 0.6 mm+quartz/sapphire wall thickness of 0.45 mm).

A micro-controller 214 was programmed to a specific scan length (11 mm) and speed (87 mm/s) for the servo motor 216. The microcontroller 214 was programmed with code to control the motor 216 to sweep back and forth over an angle of 45° with a 2.5 ms delay on either end. The motor 216 used 4.8 V, giving 1.8 kg-cm in stall torque at 0.10 s per 60°. The fiber 208 was threaded through and locked down onto an arm attached to the motor 216. The motor 216 was powered by a battery pack 218, with a circuit board 220 enabling an external on/off switch. The lower jaw comprised steel tubing supporting the quartz or sapphire square tubing. Blood vessels were compressed onto the quartz/sapphire chamber using a glass microscope slide 206, simulating a transparent upper jaw.

Burst pressure (BP) measurements were taken. Vessel BP measurements are a standard method for determining vessel seal strength. The setup included a pressure meter, infusion pump, and iris clamp. The vessel lumen was clamped over a cannula attached to the pump. Deionized water was flowed at 100 ml/hr and maximum BP recorded. A successful seal exceeded 360 mmHg, or three times systolic blood pressure (120 mmHg), consistent with industry standards for destructive testing of vessel seals.

A two-tailed student's t-test was used to determine differences between the quartz and sapphire chamber data groups for the following parameters: vessel diameter, burst pressure, internal peak temperature, external peak temperature, internal cooling time, and external cooling time. A value of P<0.05 was considered to be statistically significant between data sets.

### Computational Simulations

Optical and thermal simulations were performed to further characterize example implementations of the end effector assemblies described in this document. Zemax and Monte Carlo optical simulations were performed.

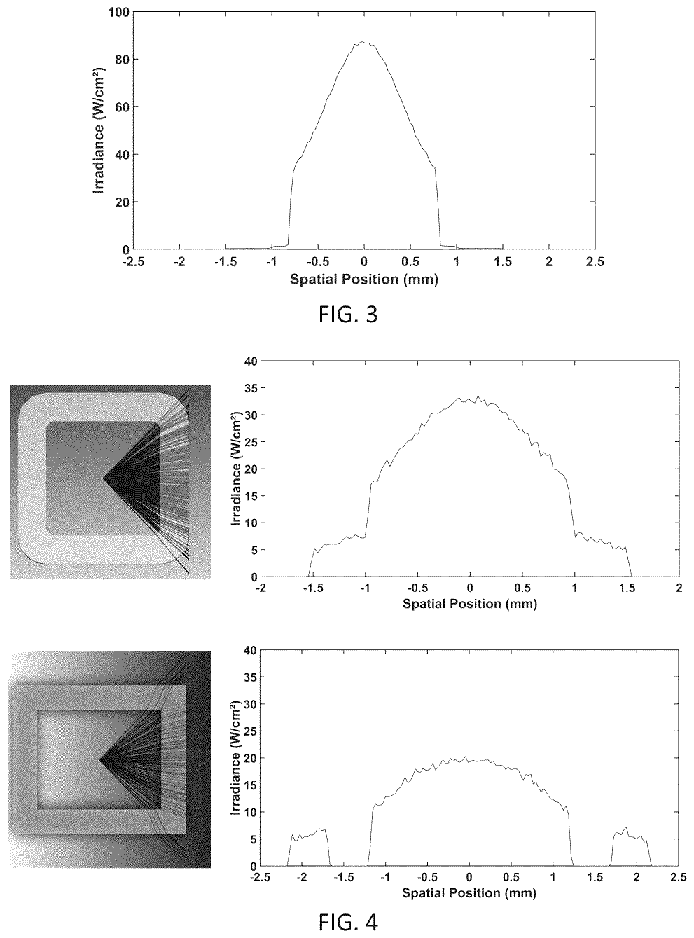

In the Zemax simulations, quartz and sapphire chambers were designed using identical dimensions to the experiments. A point source was used for simulations. FIG. 3 is a graph of irradiance versus spatial position (mm) that illustrates the initial laser beam profile exiting the optical fiber, used for both the quartz and sapphire Zemax simulations.

FIG. 4 shows the spatial distribution of the laser beam exiting the chamber for quartz and sapphire. FIG. 4 shows ray tracing showing both (left column) beam divergence through the optical chamber and (right column) initial beam profile, for both quartz (top row) and sapphire (bottom row), using Zemax optical software. Note that the quartz chamber had curved edges, due to commercial availability of square quartz tubing, while the sapphire chamber was assembled from four individual optical windows, resulting in straight edges, due to the lack of commercial availability for square sapphire tubing. In general, the end effector assemblies described in this document can have chambers of any appropriate shape, e.g., with curved or straight edges.

The wider output beam profile of quartz (3.2 mm) compared to sapphire (2.5 mm) is responsible for the larger seal zone observed. The larger seal zone (with higher irradiance in center) may enable simultaneous bisection of the vessel with thermal sealing (from lower irradiation at the ends) of both vessel ends, in a one-step process. When using a sapphire chamber with a smaller output beam profile, the seal zone was narrower, which may prohibit simultaneous bisection and sealing of vessels. This simulation result also explains the narrow carbonization zone at the center of the seal using a quartz chamber. The small peaks in the output beam profile for the sapphire tubing are due to the refraction of light rays at the straight corners of the tubing.

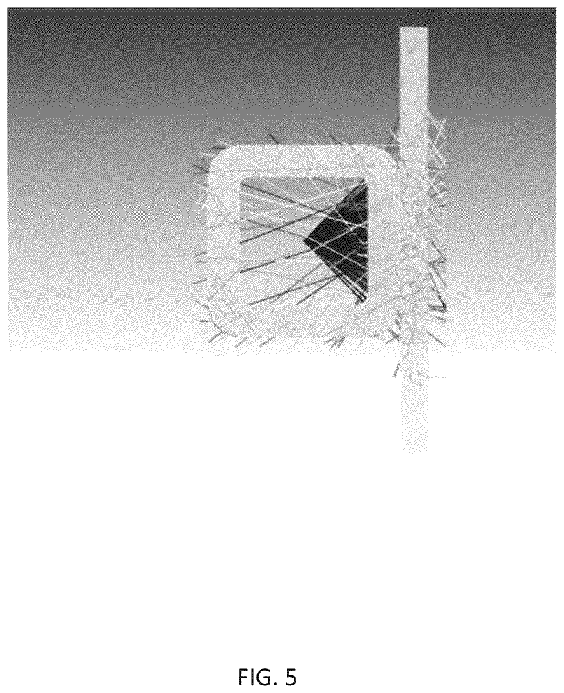

Monte Carlo (MC) simulations were also performed (using Zemax software) to determine the amount of light reflected and scattered back into the quartz chamber. FIG. 5 illustrates Monte Carlo simulations showing light transport through the quartz chamber and into the tissue layer. A total of 1 million light rays were used in the simulations.

A Henyey-Greenstein bulk scattering model was used. Values for mean free path, transmission fraction (albedo), and anisotropy factor, g, were entered into Zemax for quartz. Mean free path and transmission factor were calculated using the following formulas: Mean free path=1/(μs+μa) and Transmission fraction=μs/(μa+μs). The following optical properties for renal arteries were used, taken from previous literature [6], μs=267 cm⁻¹, μa=20.4 cm⁻¹, and g=0.875. Using the formulas above, Mean free path=0.035 and Transmission fraction=0.929.

In the computational setup, two detectors (inside and outside the quartz chamber) were strategically positioned to measure power leaving the tissue and power re-entering the chamber, due to both Fresnel reflections and diffuse reflection (back-scattered photons). A 30 W point light source was used for simulations, similar to experiments, and 1 million rays were launched into the tissue. The external detector measured 2.63 W, or 8.7% of input power. The internal detector measured 1.97 W, or 6.6% of input power. These results were consistent with predominantly forward-scattering behavior of tissue that has a high anisotropy factor (g=0.875). Hence, to replicate experimental conditions in COMSOL simulations for thermal analysis of the quartz chamber, a reflection coefficient of 0.066 (or 6.6%) was employed for the surface.

For the thermal simulations, the heat source in the quartz chamber is provided by the following equation in COMSOL:

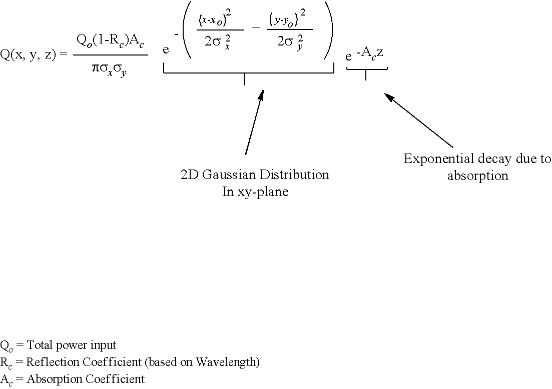

Heat flux is Q=h(Tₑₓₜ−T) where h=heat transfer coefficient of air that depends on initial material temperature (T) and surrounding medium, (e.g. air temperature, Tₑₓₜ).

A quartz chamber was designed in COMSOL using dimensions similar to experiments. The simulation incorporated a laser (heat source) directed towards the internal bottom surface of the quartz chamber, mimicking conditions encountered in the experimental setup. The laser beam was also visible on the top surface due to reflection from the bottom surface. This enabled tracking of the laser beam movement within the quartz chamber. To replicate the reciprocating motion of the fiber tip within the quartz chamber, the location of the light source was variable only along the y-axis using the interpolation feature in COMSOL, covering a scan length of 11 mm.

A domain point probe was placed on the top surface within the scan length of the light source. This probe effectively replicated the position of the thermocouple used in the experiments to measure the temperature along the scan length of the fiber tip on the top surface. After 5 s, the laser was turned off, but the simulation continued recording surface temperatures of the quartz chamber until body temperature (37° C.) was reached. Convective cooling from air played a major role in lowering the quartz chamber's temperature.

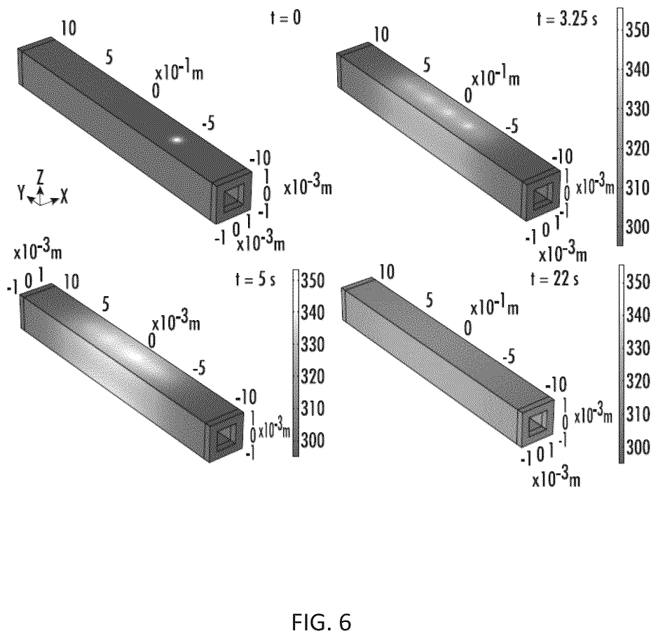

FIG. 6 shows images of the quartz chamber design and thermal simulation results at several time points (t=0, 3.25, 5, and 22 s). In particular, FIG. 6 shows COMSOL simulations showing the temperature profile on the external surface of the quartz chamber (with plugs inserted on both ends) at different time points of t=0 s (start of laser irradiation), t=3.25 s (during laser irradiation), t=5 s (after laser de-activated), and t=22 s (after 17 s of cooling).

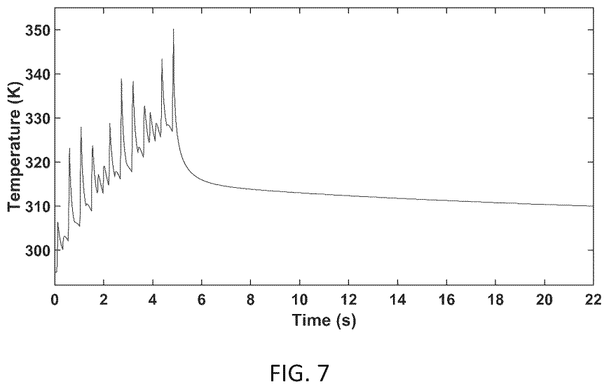

FIG. 7 shows simulated results for the temperature-time response on the external surface of the quartz chamber. External temperature (in Kelvin) of the quartz chamber as a function of time. Laser is activated at t=0 s and de-activated at t=5 s. The highest temperature was 351 K (77.85° C.), with cooling time of 16 s to reach body temperature (37° C.), consistent with experimental results.

The highest temperature was 351 K (77.85° C.), with cooling time of 16 s to reach body temperature (37° C.), again consistent with experimental results. The quartz chamber experienced a sharp temperature decrease after 5 s when the laser was turned off, due to efficient heat transfer between the quartz wall and air, facilitated by the temperature gradient. As the temperature of the quartz wall approached that of ambient air temperature, the rate of heat transfer decreased, due to the reduced temperature difference between the quartz wall and the air.

FIG. 7 shows multiple peaks and valleys at specific time intervals. When the sensor and scanned laser beam locations coincided, a temperature peak resulted due to localized thermal accumulation induced by the laser. When the laser beam was further away from the sensor, the temperature decreased. As the sensor recorded the temperature measurement when the laser and sensor are again at the same location, subsequent temperature peaks continue to increase due to thermal buildup from previous laser heating. Furthermore, the absence of peaks and valleys in the data during the cooling phase also suggests that these fluctuations arise from interplay between the sensor and scanned laser during data acquisition.

### Experimental Results

Side-firing optical fibers polished at a 50° angle delivered 94% of light sideways at a 90° angle, with 3.7% in opposite direction (−90°) and 2.3% in forward (0°) direction. Total Fresnel reflection losses for the two quartz/air interfaces measured 6%, with 94% of the side-firing light transmitted through the quartz tubing wall. These values are consistent with the refractive index mismatch between quartz at 1470 nm (n_{q}=1.445) and air (nₐ=1.0), which yields a reflection loss of 3.3% per a surface, or total of 6.6% for two surfaces.

The maximum temperatures measured on the internal surface of the quartz chamber (45.4±5.8° C.) were significantly lower than on the external surface (73.8±8.4° C.) (P=2E−4). The maximum temperatures measured on the internal surface of the sapphire chamber (66.1±15.5° C.) were not significantly different than on the external surface of the chamber (72.8±9.8° C.) (P=0.20). There was also no significant difference between the external temperatures for quartz and sapphire chambers (P=0.78).

However, the largest difference between the quartz and sapphire chamber studies was found in the cooling times. The cooling times for the external surface of the chamber are of interest, since they determine how long the surgeon must wait in between successive surgical applications of the laparoscopic sealing device, to prevent thermal damage to adjacent tissues through contact with the device. The external surface of the quartz chamber cooled down to body temperature (37° C.) in 13±4 s, while the external surface of the sapphire chamber required twice as long to cool down, at 27±7 s (P=1 E-6). There was an even larger difference in cooling times for the inside of the quartz and sapphire chambers, 4.2±3.8 s and 40.0±3.8 s, respectively (P=1 E-18), due to thermal buildup inside the sapphire chamber from higher Fresnel reflection losses at the air/sapphire interface.

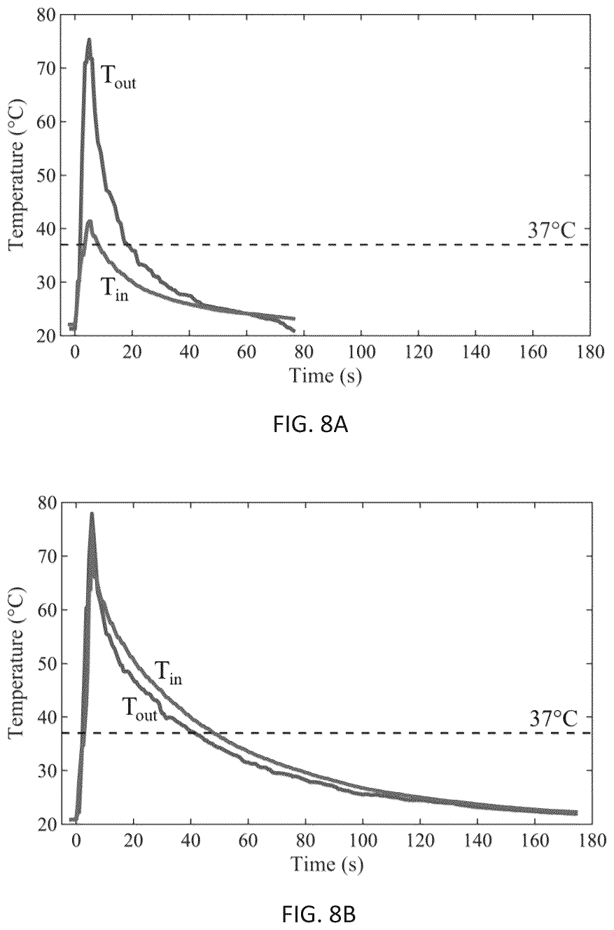

FIGS. 8A-8B show representative temperature-time data for TCs placed on the external and internal surfaces of the quartz/sapphire chambers. FIGS. 8A-8B show representative thermocouple temperature measurements on the internal (Tᵢₙ) and external (Tₒᵤₜ) surfaces of the (FIG. 8A) quartz and (FIG. 8B) sapphire optical chambers, as a function of time. Vessel sealing studies were performed with an incident laser power of 30 W at the tissue surface and a laser irradiation time of 5 s at a wavelength of 1470 nm.

Blood vessel burst pressures (BP) were measured. For the quartz chamber, vessel BP averaged 883±393 mmHg, with 13/13 vessels (100%) achieving successful BPs above 360 mmHg. For the sapphire chamber, vessel BPs measured 412±330 mmHg, with only 10/14 vessels (64%) sealed successfully. This difference in burst pressures was statistically significant (P=0.003).

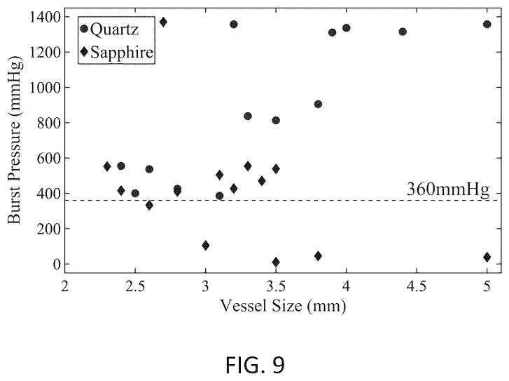

FIG. 9 shows a scatter plot of BPs as a function of vessel diameter for quartz and sapphire chambers. The dashed horizontal line shows the industry standard threshold of 360 mmHg (three times systolic blood pressure) for designation of a successful seal. For the quartz chamber, vessel BP averaged 883±393 mmHg, with 13/13 vessels (100%) recording BPs above 360 mmHg. For the sapphire chamber, vessel BPs measured 412±330 mmHg, with 10/14 vessels (64%) sealed.

It is common to observe a wide range in the vessel burst pressures of successful seals during laboratory testing, with all energy-based devices, due in part to differences among samples in vessel diameter, collagen/elastin content, attached fatty tissue layers, and water content.

As vessel diameter increases, the BPs achieved using the quartz chamber trend higher, while BPs for sapphire chamber trend lower. The decreasing trend in BPs for larger vessels treated with the sapphire chamber can be explained by observed incomplete, less than full thickness seals, due to more significant heating on the front surface of the vessel sample. Thermal coagulation of soft tissues is well known to result in dynamic changes in the optical properties of tissues, specifically an increase in light scattering, which in turn results in decreased optical penetration depth and an even steeper temperature gradient with depth. The vessel sealing process for the sapphire chamber may therefore be dominated by thermal conduction from the front surface, rather than uniform deposition of optical energy into the tissue, with this effect enhanced by the higher thermal conductivity for sapphire than quartz, as discussed further below.

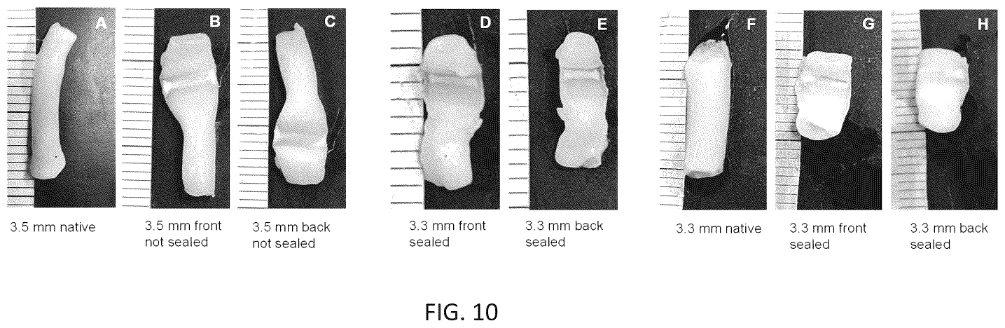

FIG. 10 shows representative blood vessels after laser treatment, for quartz and sapphire chambers. A relatively uniform and well delineated zone of thermally coagulated tissue is observed on both the front and back surfaces of blood vessels successfully sealed using both the quartz and sapphire optical chambers. However, in the vessels that failed using the sapphire chamber, as determined by low burst pressures (<360 mmHg), an incomplete thermal coagulation zone is observed on the back side of the vessel, indicating a less than full thickness seal.

FIG. 10 includes photographs of the vessels before and after sealing for sapphire and quartz. (A,B,C) A 3.5 mm diameter vessel in its native state, as well as after it was sealed unsuccessfully using the sapphire chamber, showing its front side, and back side, respectively. (D,E) A 3.3 mm diameter vessel, showing its front and back sides, respectively, after a successful seal (BP=554 mmHg) using the sapphire chamber. (F,G,H) A 3.3 mm diameter vessel in its native state, as well as after it was sealed successfully (BP=776 mmHg) using the quartz chamber, showing its front side and back side, respectively. All vessels sealed with the quartz chamber were successful. An incomplete zone of the thermal coagulation on the backside of the vessel is observed for the failed seal using the sapphire optical chamber in image (C).

### Additional Experimental Results

Electrosurgical and ultrasonic devices are used in surgical procedures for hemostatic sealing and bisection of vascular tissues. In accordance with aspects of the presently disclosed subject matter, provided is a smaller, laparoscopic device compatible design, and an output bean profile with a simultaneous approach to sealing and bisection of vessels, with optical feedback.

A 1470-nm infrared diode laser sealed and bisected 40 porcine renal arteries, ex vivo. A reciprocating, side-firing, optical fiber, housed in a transparent square quartz optical chamber (2.7×2.7×25 mm outer dimensions), delivered laser energy over an 11 mm scan length, with a range of incident powers (41-59 W) and treatment times (5-21 s). Vessel diameters ranged from 2.5-4.8 mm. Vessel burst pressure measurements were performed on each cut end (n=80) with success indicated by pressures exceeding 360 mmHg.

All vessel ends were successfully sealed and bisected (80/80). The highest incident power, 59 W, yielded short treatment times of 5-6 s. Peak temperatures on the external chamber surface reached 103° C. Time to cool down to body temperature measured 37 s. Infrared lasers simultaneously seal and bisect blood vessels, with treatment times comparable to, and temperatures and cooling times lower than reported for conventional devices.

A compact, square quartz optical chamber (2.7×2.7×25 mm OD) was used in accordance with the presently disclosed subject matter. Objectives of the following Examples include: (1) investigating the feasibility of simultaneous IR laser vessel sealing and bisection in a one-step approach, using a quartz optical chamber suitable for integration into a laparoscopic device, and (2) testing the feasibility of using the optical signal originating from the therapeutic laser and transmitted through the cut vessel, as a closed-loop, optical feedback system for immediately deactivating the IR laser upon successful vessel bisection.

### Methods Employed in Examples

Tissue Preparation. Fresh porcine kidney pairs were acquired from an abattoir (Animal Technologies, Tyler, TX). Renal arteries were dissected and stored in physiological saline in a refrigerator prior to use. A total of n=10 blood vessels, each with a similar uncompressed mean diameter of 3.2±0.5 mm, 3.4±0.6 mm, 3.4±0.8 mm, and 3.3±0.7 mm, were selected for each group of four laser power settings (41, 47, 53, and 59 W), respectively (P=0.84).

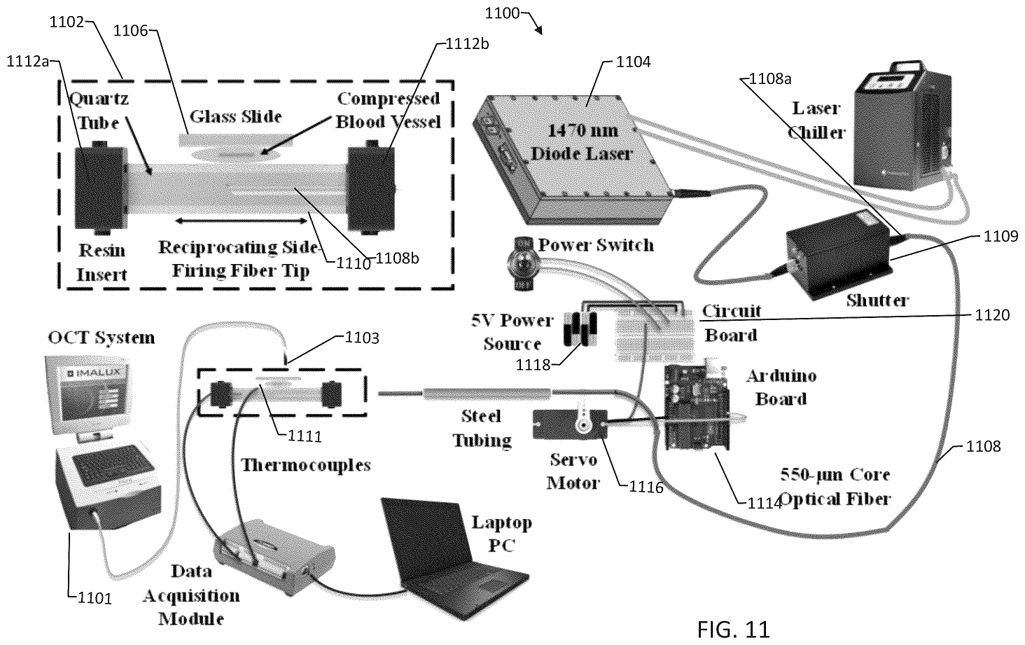

Laser Parameters. A 100-Watt, 1470-nm wavelength, IR diode laser (BrightLase, QPC Lasers, Sylmar, CA) was used for the vessel sealing studies. The laser was operated in continuous-wave (CW) mode with incident power of 41, 47, 53, and 59 W and irradiation time ranges of 20-21, 15-17, 10-11, and 5-6 s, respectively. A laser chiller was also provided. Laser power output was calibrated using a power meter (EPM1000, Coherent, Santa Clara, CA) and detector (PM-150, Coherent). A high-power fiber optic shutter (SH-200-55-1470-M-O-T-BH-SP, Oz Optics, Ottawa, Canada) was connected by a fiber optic patch-cable on one end to the laser diode module, and the other end to a surgical fiber (FIG. 11). The shutter enabled ramp up of the laser power output prior to initiating the procedure as well as stable power output during laser irradiation. Losses at multiple optical component interfaces (e.g. fiber/air interfaces) and coupling through the fiber optic shutter, limited maximum power output through the surgical fiber to 59 W.

Referring to FIG. 11, an example system 1100 for testing an example implementation of the end effector 1102 similar to the system 200 shown in FIGS. 2A and 2B is shown. The laser 1104 was connected to a side-firing fiber 1108. Two mounts (not shown) prevented the fiber 1108 from rotating. An Arduino board 1114 was pre-programmed to a specific scan length and speed for the servo motor 1116. The fiber 1108 was threaded through and locked down onto an arm attached to the servo motor 1116. The servo motor 1116 was battery powered, with an external ON/OFF switch. The lower jaw included steel tubing attached to quartz square tubing (25-mm-long with 2.7×2.7 mm OD and 1.8×1.8 mm ID). Black resin plugs 1112b, 1112a sealed the optical chamber 1110 on proximal and distal ends, with small holes on each end for insertion of the bare optical fiber 1108 (with jacket removed), and insertion of the micro-thermocouple, respectively. The black plug 1112a on the distal end additionally served to absorb stray light in the forward direction. Vessel samples were compressed onto the quartz tubing using a glide slide 1106, simulating a transparent upper jaw.

Blood vessel samples were compressed and fixed in place using a 0.5-mm-thick optical window locked in a clamp, to simulate a transparent surface for the upper jaw (FIG. 11). An optical coherence tomography (OCT) system (Niris, Imalux, Cleveland, OH) 1101 with 2.7-mm-OD probe 1103 provided non-invasive measurement of the compressed vessel thickness to confirm consistent pressures and reproducible measurements between samples. Compressed tissue thickness was fixed at 0.4 mm to closely match the optical penetration depth of IR light in water-rich soft tissues at 1470 nm, and to provide uniform, full-thickness seals.

The OCT probe 1103 was placed next to the sample and optical window 1106, to non-invasively image the tissue thickness, and then moved away before laser activation. The OCT system 1101 operated at 1310 nm, with axial and lateral resolutions of 11 and 25 μm. Images had 1.6 mm and 2.0 mm axial and lateral dimensions. The combined thickness of the compressed tissue (0.4 mm) plus optical window (0.5 mm) was less than the OCT axial scan depth of 1.6 mm, enabling measurement of compressed vessel thickness.

Side-firing Fiber Preparation. A low-OH, silica optical fiber 1108 (FG550LEC-CUSTOM, Thorlabs, Newton, NJ) with 550-μm-core, 600-μm-cladding, 1040-μm-jacket, and numerical aperture of 0.22 was used. The proximal fiber tip 1108a with high-power SMA905 connector was attached to the shutter 1109. The side-firing, angled distal fiber tip 1108b was prepared using a fiber optic polisher (Radian™, Krelltech, Neptune City, NJ), to achieve a 50° polish angle and 90° light delivery.

Fixed Optical Chamber Assembly. The optical chamber 1110 included a quartz (Technical Glass Products, Painesville Township, OH) square tubing with dimensions of 1.8×1.8 mm ID, 2.7×2.7 mm OD, 25 mm length, and 0.45 mm wall thickness. A 3D-printed, black resin plug 1112a-b (RS-F2-GPBK-04, Formlabs, Durham, NC) was placed on each end of the chamber (FIG. 11). The proximal plug 1112b had a small hole to allow insertion of the fiber 1108. The distal plug 1112a also had a small hole to allow insertion of a thermocouple (TC) 1111, but otherwise provided fluid-tight closure and absorbed stray light in the forward direction. The fiber 1108 was inserted into the quartz tubing and clamped in place, leaving a 0.6 mm space between the fiber tip and the inner walls of the tubing. The distance from fiber tip to vessel wall measured 1.05 mm (air gap of 0.6 mm+quartz wall thickness of 0.45 mm).

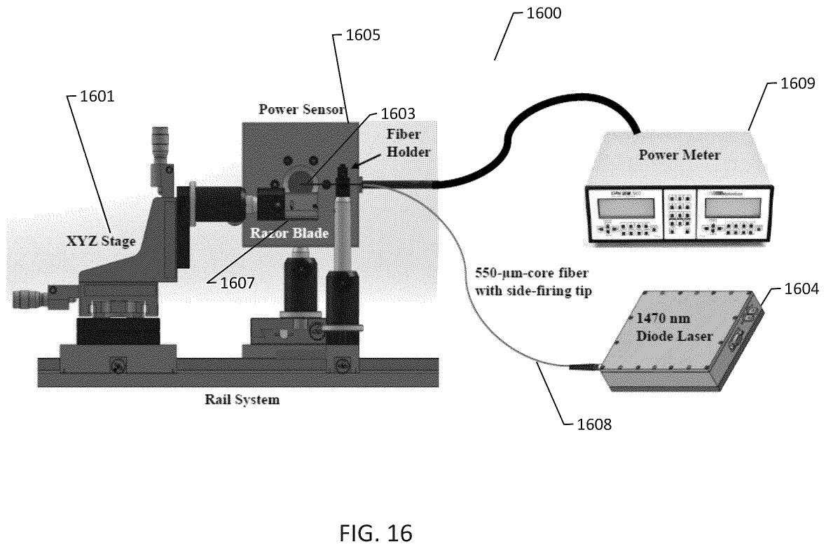

Optical Beam Characterization. Referring to FIG. 16, an example system 1600 for optical beam characterization is shown. The side-firing fiber output beam was directed towards a detector 1605 (PM100-19C, Coherent) connected to a power meter 1609 (EPM2000, Coherent). An XYZ stage 1601 (460A-XYZ, Newport, Irvine, CA) with mounted razor blade 1607 was used. To accurately simulate the distance from fiber tip 1603 to tissue sample, the razor blade 1607 was set in front of the fiber 1608, 1 mm from its edge. The razor blade 1607 was moved across the laser beam in 100 μm increments, and power recorded at each location, for beam characterization.

Reciprocating Fiber Setup. FIG. 11 shows the reciprocating, side-firing, fiber experimental system 1100. A high-power connector on the proximal fiber end 1108a was attached to the shutter 1109. Two custom mounts (not shown) and steel tubing prevented the fiber 1108 from rotating and becoming misaligned. A micro-controller 1114 (Uno, Arduino, Boston, MA) was pre-programmed to a specific scan length (11 mm) and speed (87 mm/s) for the servo motor 1116 (SG90, Deegoo-FPV, China). The microcontroller 1114 had a custom uploaded code that allowed the stepper motor 1116 to sweep back and forth over an angle of 45° with a 2.5 ms delay between steps. The motor 1116 used 4.8 V, giving 1.8 kg-cm in stall torque at 0.10 s per 60°. The fiber 1108 was threaded through and locked down onto an arm attached to the motor 1116. The motor 1116 was powered by a battery pack 1118, with a circuit board 1120 enabling an external on/off switch. Blood vessels were compressed onto the quartz chamber using a glass microscope slide 1106 as an optical window, simulating a transparent surface for the upper jaw.

Temperature Measurements. Two micro-thermocouples 1111 (5TC-TT-T-36-72, Omega, Norwalk, CT) with 125-μm-OD were used: one TC was placed inside the quartz and another TC placed in contact with the chamber's external surface on the opposite side to the exiting laser beam. The TC tip extended 1 mm beyond the plug, within the tubing, but beyond the laser beam path. A personal computer with temperature software (TracerDAQ+InstaCal, Omega) recorded TC temperatures as a function of time (FIG. 11).

Burst Pressure (BP) Measurements. The vessel burst pressure setup for measuring vessel seal strengths included a pressure meter (717 100 G, Fluke, Everett, WA), infusion pump (78-01000C, Cole Parmer, Vernon Hills, IL), and iris clamp (ID25, Thorlabs) [3-11]. The vessel lumen was clamped over a cannula attached to the pump. Deionized water was flowed at 100 ml/hr and maximum BP recorded. A successful seal exceeded 360 mmHg, or three times systolic blood pressure (120 mmHg), consistent with industry standards for testing.

Optical Transmission Measurements. A similar 550-μm-core optical fiber as for the IR laser sealing/cutting experiments, was also used for optical transmission measurements, but the bare distal fiber tip was instead polished flat. A total of 8 blood vessels (d=3.1±0.6 mm) were tested. The IR laser was operated in CW mode at an incident power of 6.5 W, producing a circular beam diameter of 0.560 mm, and yielding an irradiance of 26.4 W/mm² or 2640 W/cm², at the vessel surface. The working distance between the fiber tip and tissue surface was kept fixed at 1.3 mm. The tissue was compressed between two acrylic windows to a 0.4 mm thickness. A 1.5-mm-diameter hole was drilled in the center of each acrylic window, to enable the optical signal to be transmitted directly through the tissue to the detector, without additional Fresnel reflection losses.

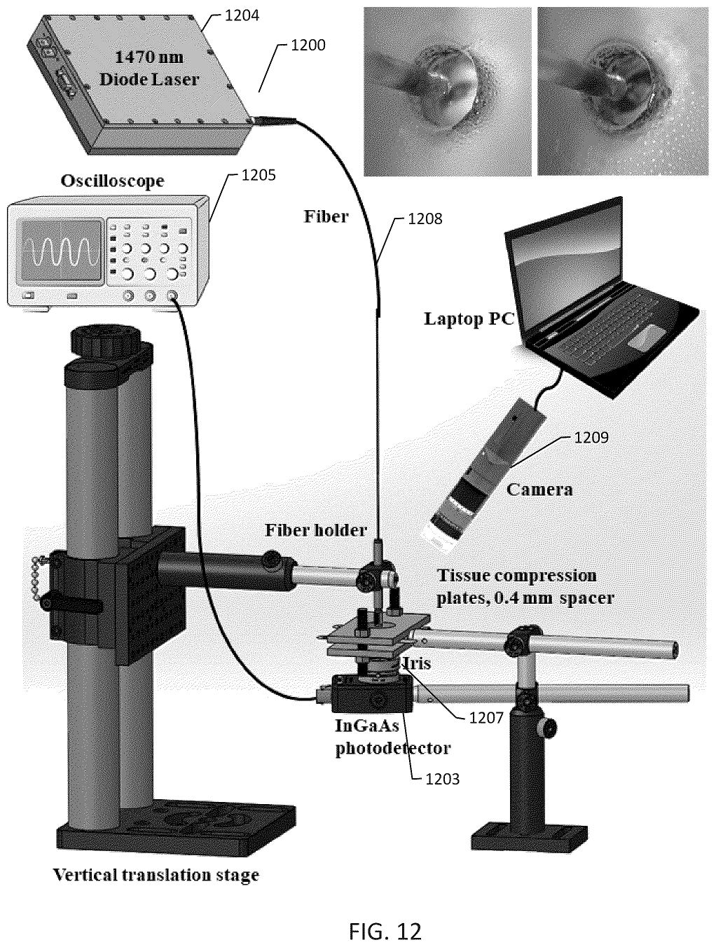

FIG. 12 shows an example experimental system 1200 for acquiring the optical transmission signal during continuous-wave laser ablation of compressed porcine renal blood vessels. Left top inset—start of carbon layer formation on the tissue surface during laser irradiation. Right top inset—formation of an ablation crater during laser irradiation. The optical transmission signal was acquired by an InGaAs photodiode detector 1203 (PDA400, Thorlabs) connected to an oscilloscope 1205 (TDS 2002b, Tektronix, Beaverton, OR). A neutral density filter (not shown) with an optical density of 4.0 (NE40, Thorlabs) was placed between the tissue and photodetector 1203 to attenuate the signal and protect the photodetector 1203. An iris 1207 (SM1 D12C, Thorlabs) with 1.5-mm-diameter opening was also placed in front of the photodetector 1203 to reduce contributions from multiple scattered light to the transmission signal. The laser 1204, which is connected to fiber 1208, was de-activated once the oscilloscope signal saturated, corresponding to an ablated hole in the tissue. A compact camera 1209 (AF4915ZT, Dino-lite, Torrance, CA) recorded video of each experiment.

### Optical Characterization of Side-Firing Fiber

Side-firing optical fibers polished at a 50° angle delivered 94% of light sideways at a 90° angle, with 2.3% in the forward (0°) direction and 3.7% in the −90° direction. Total Fresnel reflection losses for the two quartz-air interfaces measured 6.6%, with 93.4% of the side-firing light transmitted through the quartz tubing wall, as also described elsewhere herein. A razor blade scan was performed at a distance of 1 mm from the fiber, corresponding to where the blood vessel sample would be located on the external surface of the quartz tubing. The 1/e² beam spot size measured 600×800 μm.

Using the elliptical beam dimensions of 0.06×0.08 cm at the tissue surface, scanned over a length of 1.1 cm, it is possible to estimate the total fluence (J/cm²) delivered to the tissue by the reciprocating beam, for each incident power and irradiation time. An average uncompressed vessel diameter of about 0.33 cm used in these studies, and a typical 50% increase in vessel width with compression previously reported by several research groups [12,13] to 0.50 cm, yields a correction factor of (0.50/1.1), or 0.45. Thus, the laser beam scan length of 1.1 cm is only incident on the compressed tissue sample width of 0.5 cm, 45% of the time. The rectangular beam area for the entire scan length is given by the measured beam width (0.08 cm) times laser scan length (1.1 cm), or 0.088 cm². Hence, total fluence (F) delivered to the vessel for each laser power group and fluence level is calculated to be:

$$\begin{matrix}F = [ ( 0.45 ) ( 41 W ) ( 20.5 s ) ] / 0.088 {cm}^{2} = 4 , 298 J / {cm}^{2} \\ F = [ ( 0.45 ) ( 47 W ) ( 16 s ) ] / 0.088 {cm}^{2} = 3 , 845 J / {cm}^{2} \\ F = [ ( 0.45 ) ( 53 W ) ( 10.5 s ) ] / 0.088 {cm}^{2} = 2 , 846 J / {cm}^{2} \\ F = [ ( 0.45 ) ( 59 W ) ( 5.5 s ) ] / 0.088 {cm}^{2} = 1 , 659 J / {cm}^{2}\end{matrix}$$

This large difference in total fluence can be explained by the continual loss of heat through thermal conduction during the longer laser irradiation durations, thus requiring greater total fluence to achieve the threshold temperatures needed for tissue ablation. Note that thermal conduction of heat during laser irradiation is also desirable as it contributes to thermal sealing of the vessel.

### Burst Pressure (BP) Measurements

Vessel burst pressures (mmHg) were conducted for all vessels sealed and bisected (n=40) at four different laser incident power levels and irradiation times. All vessel cut ends tested (80/80) withstood BPs above three times systolic pressure (360 mmHg), yielding a 100% success rate. The shortest irradiation time was 5-6 s at 59 W. The BP data is summarized in Table 1.

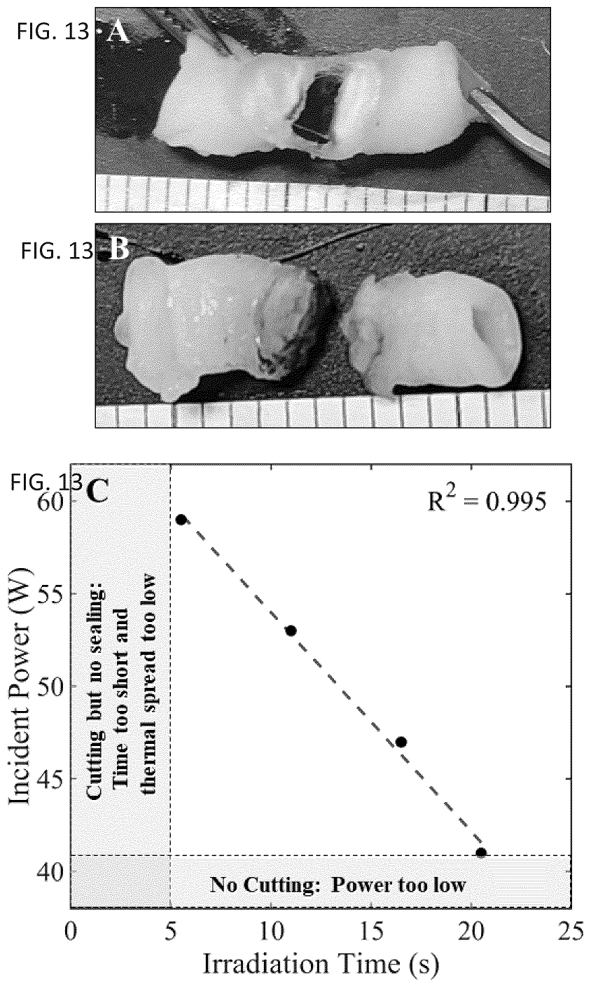

Representative images of a blood vessel sample before and after bisection are shown in FIGS. 13A and 13B. Laser irradiation times shorter than 5 s resulted in tissue cutting, but not sealing, presumably due to insufficient thermal conduction and thermal spread during the laser treatment time. Laser incident powers less than 41 W failed to produce a full-thickness cut, due to insufficient irradiance. Otherwise, there was a strong linear fit to the power/time data points, demonstrating that higher laser incident power enables shorter laser irradiation times, as expected (FIG. 13C).

### Thermal Characterization of Quartz Jaw

Temperature-time data for micro-thermocouples placed on the external (Tₒᵤₜ) and internal (Tᵢₙ) surfaces of the quartz chamber was also collected using the shortest irradiation time of 5-6 s and incident power of 59 W. The maximum temperature measured on the internal chamber surface, 77° C., was lower than on the external surface, 103° C. Cooling times for the external chamber surface are of much interest, since they determine how long the surgeon must wait in between successive activations of the laparoscopic device. The external surface of the quartz chamber cooled to body temperature (37° C.) in 37 s.

### Optical Transmission Signal During Blood Vessel Ablation

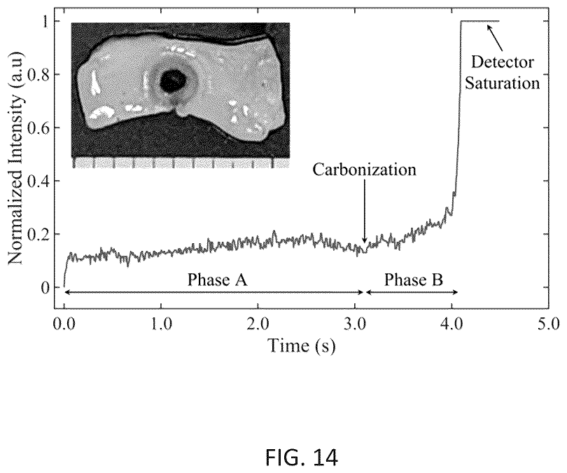

Optical transmission measurements revealed two distinct phases (FIG. 14). Phase A is the period between the start of CW laser irradiation, with corresponding dehydration and coagulation of the tissue, and the beginning of carbon formation on the vessel surface. Analysis of the videos showed that the tissue surface discolored due to shrinkage from water evaporation and thermal denaturation. During phase B, tissue carbonization begins to form and grow in size. A rapid increase in optical transmission and eventual saturation of the signal occurs, as the tissue is vaporized and a full-thickness ablation crater forms. The laser was de-activated when the signal saturated. This steep and rapid rise in signal at the photodetector, corresponding to tissue perforation, can serve as a potential optical feedback system during laser bisection of vessels. Calculations for the ablation velocity, based on these experiments, are provided below.

### Vessel Sealing and Bisection Studies

Previous benchtop studies were performed on a large scale, with bulk optical components unsuitable for integration into standard laparoscopic devices. These previous studies also utilized a two-step process with sequential sealing and then cutting of the vessel, involving a more complex method of translation of the optical components to change the beam focus and laser spot size [4]. This study investigated the feasibility of simultaneous IR laser vessel sealing and bisection of porcine renal arteries in a one-step approach, using a compact quartz optical chamber suitable for integration into the distal end effector jaws of standard 5-mm-OD laparoscopic devices.

It should be noted that previous computer simulations of the laser irradiation distribution through the quartz chamber disclosed elsewhere herein, showing a Gaussian-like beam profile, with additional steps on the wings, motivated this approach to simultaneous sealing and bisection of vessels, based on the ability to coagulate and seal at the periphery of the beam and ablate or cut in the center of the beam. Laboratory studies set forth herein above also demonstrated successful sealing of blood vessels at 30 W for 5 s at 1470 nm. These parameters were therefore chosen as a baseline, and the laser power was elevated in steps with different irradiation times, and BPs were measured to confirm success.

Table 1 shows the shortest irradiation times required at each power level to simultaneously seal and bisect the vessel. It should be noted that the irradiation times contain a small range rather than a singular value due to several factors, including variable amount of fat and water content in vessels, different amounts of collagen/elastin levels (C/E ratio) and variable vessel sizes. Larger blood vessels had thicker walls which made it more difficult for the chamber to create equal pressure across the vessel and to efficiently compress it to the desired 0.4 mm thickness to match the optical penetration depth of the 1470-nm laser energy in tissue. These factors also explain in part the large range of BPs measured in Table 1.

TABLE 1
Burst pressures (BP) of both bisected segments (S1/S2) of
blood vessels for each incident power and irradiation time.

| Incident | Irradiation | |
|---|---|---|
| Laser Power | Time (s) | Mean BP (mmHg) |
| 41 | 20-21 | S1: 788 ± 374/S2: 758 ± 320 |
| 47 | 15-17 | S1: 1046 ± 355/S2: 1053 ± 320 |
| 53 | 10-11 | S1: 1199 ± 149/S2: 875 ± 421 |
| 59 | 5-6 | S1: 984 ± 351/S2: 976 ± 350 |

It should be emphasized that several previous studies have also reported wide ranges in BP values using RF, US, and IR sources [6, 14-16]. In this study, the lowest laser irradiation time in each range provided a complete cut, but with a small amount of fascia remaining, easily manually pulled apart with tweezers in the laboratory or the device jaws in a clinical situation (FIG. 13A). The highest irradiation time within each range provided a complete cut with no remaining fascia strands (FIG. 13B).

The highest incident power, 59 W, yielded treatment times of 5-6 s, comparable to conventional RF and US devices. For example, a recent study reported energy activation durations of 7.7 s and 7.9 s for US and RF devices, respectively [17].

Peak temperatures and cooling times for the external surface of the chamber are also important, since they determine the safety profile of the device as well as the time the surgeon must wait between successive activations of the laparoscopic device. Peak temperatures measured 103° C. on the outside of the quartz chamber at 59 W and 5-6 s. Furthermore, the external surface of the chamber cooled to body temperature (37° C.) in 37 s. These values also appear promising, when directly compared with values reported for conventional RF and US devices. For example, a recent study reported that median external jaw temperatures were 126° C. for RF devices and 218° C. for US devices [17]. The quartz chamber's cooling time of 37 s also compares favorably with reported mean cooling times of 54 s and 68 s for two different RF devices [18].

### Optical Signal Transmission Studies

This study also demonstrated the feasibility of using the optical transmittance signal during IR laser tissue ablation as a closed-loop, optical feedback system for immediately deactivating the laser upon successful bisection. Optical transmission analysis during the ablation study revealed slightly variable pre-ablation times (Phase A), due to differences in tissue size, fat content, water content and C/E levels between samples. Consequently, an optical feedback system that deactivates the laser upon ablation is desired. Phase A in FIG. 14 includes the phenomena of water absorbing laser light, followed by its evaporation and tissue coagulation, which results in the decrease of signal right before the start of phase B. This decrease is believed to be associated with a rise in the scattering coefficient of the tissue surface due to thermal coagulation, which is a commonly observed property [19].

Phase B starts with carbonization and ends when the tissue ablation is complete. The start of tissue carbonization was observed as a thin layer of blackened tissue at the surface (FIG. 12 inset). Carbonization enhances light absorption, leading to a reduction in light transmission. Nevertheless, the creation of carbonized tissue did not lead to a reduction in transmission in the forward direction. Video frames displayed a progression where initially, a limited central area of carbonized tissue was generated. This area then transformed into a growing ring, producing an ablation hole in the tissue center.

Based on these observations, we hypothesize that upon carbonization, the tissue rapidly vaporized, forming a small ablation hole because of significantly increased absorption. As the carbonized zone expanded outward in the form of a growing ring, the hole diameter increased (FIG. 12). This sequence of events could potentially explain the absence of any observed decline in light transmission, as reported in previous studies [20]. Consequently, when the tissue is completely vaporized and the hole is complete, the signal saturates, and the laser can be de-activated (FIG. 14).

### Example Device Design

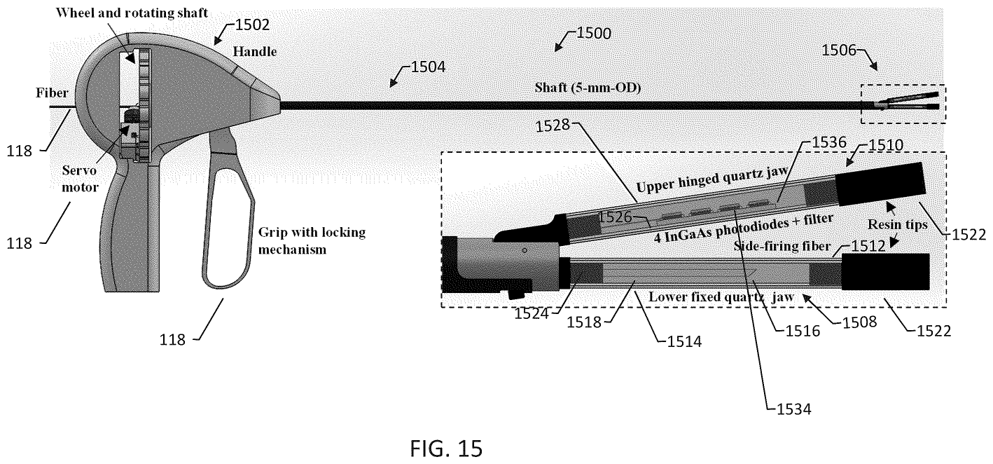

FIG. 15 shows a diagram of an example embodiment of a laparoscopic device 1500 of the presently disclosed subject matter, integrating the transparent quartz optical chamber as disclosed herein. The device includes a handle 1502, an elongated body 1504 extending from the handle 1502, and an end effector assembly 1506 secured to a distal portion of the elongated body 1504. The handle 1502 includes one or more control interfaces configured to manipulate the end effector assembly 1506. The control interfaces can include, for example, a movable handle, a trigger, a switch, and a button. The handle 1502 can include a wheel, servo motor, or rotation control configured to rotate the elongated body 1504, and the end effector assembly 1506, relative to the handle 1502.

The inset in FIG. 15 illustrates the end effector assembly 1506 in detail. End effector assembly 1506 is similar to the end effector assembly 106 show in FIG. 1B. The end effector assembly 1506 comprises a first jaw member 1508 and a second jaw member 1510. The first jaw member 1508 includes a transparent tissue contacting surface 1512 and at least one transparent tissue viewing portion 1514. The first jaw member 1508 includes a fluid-tight cavity 1516 to keep fluid out and configured to receive an optical fiber 1518. In some examples, the fluid-tight cavity 1516 is sufficiently sealed to be air-tight.

The transparent tissue contacting surface 1512 and the transparent tissue viewing portion 1514 can be made, for example, from quartz, sapphire, or any other appropriate material. The term “transparent” is used in this document to refer to material that substantially transmits light in the visible range of 400-700 nm for the surgeon to see through the device and in the infrared range at a wavelength suitable for sealing or cutting tissue or both. For example, the optical fiber 1518 may transmit light having a wavelength in a range of about 800 nm to about 2500 nm.

The first jaw member 1508 includes a reflector 1520 configured to reflect a substantial portion of light from the optical fiber 1518 towards the transparent tissue contacting surface 1512. In some examples, the reflector 1520 comprises a side-firing fiber tip of the optical fiber 1518, e.g., created by an angled tip. In some examples, a small mirror or other optical element is positioned within the cavity 1516 to direct light exiting the optical fiber 1518 towards the tissue contacting surface 1512.

The first jaw member 1508 can include a first opaque plug 1522 at a distal end of the first jaw member 1508. The first jaw member 1508 can include a second opaque plug 1524 at a proximal end of the first jaw member 1508. The opaque plugs 1522 and 1524 can be useful, for example, to prevent stray light from exiting the first jaw member 1508, and to close the distal tips of the optical chambers to provide fluid-tight closure.

The first jaw member can include one or more mounts 1532 configured to prevent the optical fiber 1518 from rotating within the fluid-tight cavity 1516. The mounts 1532 can be useful, for example, where the reflector 1520 is an angled fiber tip that should remain oriented towards the tissue contacting surface 1512.

The upper, movable jaw 1508, pivots on a hinge, and is used to compress the vessel. The incorporation of a transparent quartz chamber provides for the addition of photodiodes (PD) 1534, for measuring the optical transmission signal and providing optical diagnostic feedback. The quantity of PDs 1534 implemented should encompass the complete fiber scan length to ensure vessel bisection. To protect the photodiodes from excessive laser light, an absorptive neutral density filter can be employed. The bottom, fixed jaw 1508 provides therapeutic IR laser delivery, also with a transparent quartz chamber housing the reciprocating, side-firing, optical fiber for vessel sealing/bisection. The device can also involve constructing and evaluating the feedback system within a functional laparoscopic quartz jaw prototype.

In the results described above, a simple transparent optical window was used instead for compressing the tissue, and crudely simulating the optical and thermal properties of a transparent upper jaw. However, both jaws will be transparent in a final design, so this simple approach is still relevant for the optical and thermal studies.

The reciprocating fiber scan length and speed were kept constant in this preliminary study, for simplicity, and to prove feasibility of the simultaneous IR laser sealing and cutting technique. Although the parameters programmed were practical and similar to those used elsewhere herein, a further approach is to customize scan length to match the compressed vessel width. This approach will ensure that excess laser energy is not transmitted around the sample, which is not only inefficient, but may also potentially result in safety issues due to higher device jaw temperatures. More efficient, custom scan lengths may also further lower device temperatures and cooling times, as well as laser treatment times.

It is also important to emphasize that there is significant variation in blood vessel composition (e.g. collagen/elastin ratio) among samples, which factors into the results (e.g. yielding a wide range of burst pressures).

Finally, the peak temperatures measured in this study for the IR laser vessel sealing device are lower than the peak temperatures reported for conventional electrosurgical and ultrasonic devices, thus producing a better safety profile. Potential for thermal damage to adjacent healthy tissues through accidental contact with the device should be monitored.

Simultaneous IR sealing and bisection of blood vessels was conducted using a square quartz optical chamber (2.7×2.7×25 mm outer dimensions) that can be integrated into a standard 5-mm-OD laparoscopic device. The shortest irradiation time was 5-6 s using 59 W of laser power. Peak temperatures on the external chamber surface measured 103° C., with a time of 37 s to cool to body temperature. Optical transmission measurements showed a rapid increase and saturation of the photodetector signal upon tissue ablation, which can be used as an optical feedback system to confirm vessel bisection during the procedure. A depiction of the proposed laparoscopic prototype, showcasing the transparent quartz optical chamber employed in this study, was presented. Design and construction of a complete prototype device for use during in vivo studies is provided.

### Ablation Velocity

In ablation models, the postulation of energy equilibrium at the ablation front gives rise to a constant, steady-state ablation velocity [20]. The ablation velocity was determined from the moment of tissue carbonization. Hence, phase B of the transmission signal (FIG. 14), was used to calculate all ablation velocity model variables for blood vessels at a laser wavelength of 1470 nm. Average ablation velocity is approximated by the formula [21]:

$$ablation velocity v [ \frac{cm}{s} ] = \frac{{fu}_{a} dkE}{Q}$$

Where
E=irradiance [W/cm²]
Q=heat of vaporization of water [J/cm³]=2520 J/cm³
uₐ=absorption coefficient of carbonized tissue [cm⁻¹]=21.7 cm⁻¹ [21]
d=thickness of carbon layer [cm]=25 μm [20]
k=augmentation factor from multiple light passes through carbon layer due to light scattering and total internal reflection
f=apparent efficiency of converting absorbed energy into ablation=0.45 [22]

In the transmission studies, incident laser power was 6.5 W with a circular beam diameter of 0.560 mm. Irradiance (E) is calculated as:

$$E = \frac{6.5}{( 3.14 ) ( 0.28 ) 2} = 26.4 W / {mm}^{2} = 2640 W / {cm}^{2}$$

v was calculated using total time of phase B. Average time of phase B (n=8) transmission signal was 0.9 s. Consequently, for tissue compressed to 0.4 mm:

$$v = \frac{0.4}{0.9} = 0.44 mm / s = 0.044 cm / s$$

The missing variable in this model for ablation velocity of the vessel at 1470 nm is the augmentation factor, k, that was calculated using the velocity equation as:

$$k = \frac{vQ}{{fu}_{a} dE} = \frac{( 0.044 ) ( 2520 )}{( 0.45 ) ( 21.7 ) ( 0.0025 ) ( 2640 )} = 1.7$$

Unless defined otherwise, all technical and scientific terms used herein have the same meaning as commonly understood to one having ordinary skill in the art to which the presently disclosed subject matter belongs. Although, any methods, devices, and materials similar or equivalent to those described herein can be used in the practice or testing of the presently disclosed subject matter, representative methods, devices, and materials are now described.

Following long-standing patent law convention, the terms “a”, “an”, and “the” refer to “one or more” when used in this application, including the claims. Thus, for example, reference to “a vial” can include a plurality of such vials, and so forth.

Unless otherwise indicated, all numbers expressing quantities of length, diameter, width, and so forth used in the specification and claims are to be understood as being modified in all instances by the terms “about” or “approximately”. Accordingly, unless indicated to the contrary, the numerical parameters set forth in this specification and attached claims are approximations that can vary depending upon the desired properties sought to be obtained by the presently disclosed subject matter.

As used herein, the terms “about” and “approximately,” when referring to a value or to a length, width, diameter, temperature, time, volume, concentration, percentage, etc., is meant to encompass variations of in some embodiments±20%, in some embodiments ±10%, in some embodiments ±5%, in some embodiments ±1%, in some embodiments 0.5%, and in some embodiments ±0.1% from the specified amount, as such variations are appropriate for the disclosed apparatuses and devices.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, the term “and/or” when used in the context of a listing of entities, refers to the entities being present singly or in combination. Thus, for example, the phrase “A, B, C, and/or D” includes A, B, C, and D individually, but also includes any and all combinations and sub-combinations of A, B, C, and D.

### REFERENCES

All references listed below, as well as all references cited in the instant disclosure, including but not limited to all patents, patent applications and publications thereof, scientific journal articles, and database entries are incorporated herein by reference in their entireties to the extent that they supplement, explain, provide a background for, or teach methodology, techniques, and/or compositions employed herein.
[1] Blencowe N S, Waldon R, Vipond M N (2018) Management of patients after laparoscopic procedures. BMJ 360:k120
[2] Meeuwsen F C, Guedon A C P, Arkenbout E A, van der Elst M, Dankelman J, van den Dobbelsteen J J (2017) The art of electrosurgery: trainees and experts. Surg Innov 24(4):373-378
[3] Cilip C M, Rosenbury S B, Giglio N, Hutchens T C, Schweinsberger G R, Kerr D, Latimer C, Nau W H, Fried N M (2013) Infrared laser thermal fusion of blood vessels: preliminary ex vivo tissue studies. J Biomed Opt 18(5):058001
[4] Giglio N C, Hutchens T C, Perkins W C, Latimer C, Ward A, Nau W H, Fried N M (2014) Rapid sealing and cutting of porcine blood vessels, ex vivo, using a high power, 1470-nm diode laser. J Biomed Opt 19(3):038002
[5] Cilip C M, Kerr D, Latimer C A, Rosenbury S B, Giglio N C, Hutchens T C, Nau W H, Fried N M (2017) Infrared laser sealing of porcine vascular tissues using a 1470 nm diode laser: preliminary in vivo studies. Lasers Surg Med 49(4):366-371
[6] Hardy L A, Hutchens T C, Larson E R, Gonzalez D A, Chang C H, Nau W H, Fried N M (2017) Rapid sealing of porcine renal vessels, ex vivo, using a high power, 1470-nm laser and laparoscopic prototype. J Biomed Opt 22(5):058002
[7] Giglio N C, Fried N M (2021) Computational simulations for infrared laser sealing and cutting of blood vessels. IEEE J Sel Top Quantum Electron 27(4):1-8
[8] Giglio N C, Fried N M (2022) Nondestructive optical feedback systems for use during infrared laser sealing of blood vessels. Lasers Surg Med 54(6):875-882
[9] Giglio N C, Grose H M, Fried N M (2022) Comparison of fiber optic linear beam shaping designs for laparoscopic laser sealing of vascular tissues. Opt Eng 61(2):026112
[10] Saeed W M, Fried N M (2023) A real time fluorescence feedback system for infrared sealing of blood vessels. IEEE J Sel Top Quantum Electron 29(4):1-7 [11] Saeed W M, O'Brien P J, Yoshino J, Restelli A R, Traynham A J, Fried N M
(2023) Comparison of quartz and sapphire optical chambers for infrared laser sealing of vascular tissues using a reciprocating, side-firing optical fiber: simulations and experiments. Laser Surg Med 55(10):886-899
[12] Pearce J A, Thomsen S L (2000) Blood vessel architectural features and their effects on thermal phenomena. Proc SPIE 1029706
[13] Giglio N C, Grose H M, Fried N M (2022) Reciprocating side-firing fiber for laser sealing of blood vessels. Proc SPIE 11936:1-7
[14] Lamberton G R, His R S, Jin D H, Lindler T U, Jellison F C, Baldwin D D (2008) Prospective comparison of four laparoscopic vessel ligation devices. J Endourol 22(10):2307-2312
[15] Tanaka R, Gitelis M, Meiselman D, Abar B, Zapf M, Carbray J, Vigneswaran Y, Zhao J C, Ujiki M (2015) Evaluation of vessel sealing performance among ultrasonic devices in a porcine model. Surg Innov 22(4):338-343
[16] Szyrach M N, Paschenda P, Afify M, Schaller D, Tolba R H (2012) Evaluation of the novel bipolar vessel sealing and cutting device BiCision(R) in a porcine model. Minim Invasive Ther Allied Technol 21:402-407.
[17] Brecht L, Wallwiener M, Schott S, Domschke C, Dinkic C, Golatta M, Schuetz F, Fluhr H, Stenzinger A, Kirchner M, Sohn C, Rom J (2017) Implementation of a novel efficacy score to compare sealing and cutting devices in a porcine model. Surg Endosc 32(2):1002-1011
[18] Jimenez Cl, Leith A, Ringold F (2022) Novel multifunctional robotically assisted bipolar instrument for simultaneous radiofrequency sealing and transection: preclinical and single center experience. BMC Surg 22(1):1-8
[19] Muller G J, Roggan A (1995) Laser-induced Interstitial Thermotherapy. SPIE Press: Bellingham, W A.
[20] Verdaasdonk R M, Borst C, van Gemert M J C (1990) Explosive onset of continuous wave laser tissue ablation. Phys Med Biol 35(8):1129-1144
[21] Oregon Medical Laser Center website (https://omic.org/spectra/carbon/).
[22] Malskat W S J, Poluektova A A, van der Geld CWM, Neumann HAM, Weiss R A, Bruijninckx C M A, van Gemert M J C (2013) Endovenous laser ablation (EVLA): a review of mechanisms, modeling outcomes, and issues for debate. Lasers Med Sci 29(2):393-403

The presently disclosed subject matter can be embodied in other forms without departure from the spirit and essential characteristics thereof. The embodiments described therefore are to be considered in all respects as illustrative and not restrictive. Although the present subject matter has been described in terms of certain preferred embodiments, other embodiments that are apparent to those of ordinary skill in the art are also within the scope of the present subject matter.

What is claimed is:

1. An end effector assembly for a surgical instrument, the end effector assembly comprising:
a first jaw member comprising:
a transparent tissue contacting surface;
at least one transparent viewing portion;
a fluid-tight cavity configured to receive an optical fiber; and
a reflector configured to reflect a substantial portion of light from the optical fiber towards the transparent tissue contacting surface; and
a second jaw member configured to move towards the transparent tissue contacting surface of the first jaw member.

2. The end effector assembly of claim 1, wherein the first jaw member comprises a substantially rectangular tube having four sides, and wherein the transparent tissue contacting surface is a first side of the four sides and the transparent viewing portion is a second side of the four sides.

3. The end effector assembly of claim 2, wherein at least a portion of each of the four sides is transparent.

4. The end effector assembly of claim 1, wherein the first jaw member comprises a tube having a substantially circular cross-section, and wherein the transparent tissue contacting surface is a first arc segment of the tube and the transparent viewing portion is a second arc segment of the tube.

5. The end effector assembly of claim 1, wherein the reflector comprises a side-firing fiber tip of the optical fiber.

6. The end effector assembly of claim 1, comprising a motor assembly configured for moving a tip of the optical fiber within the fluid-tight cavity of the first jaw member.

7. The end effector assembly of claim 6, wherein the motor assembly comprises a servo motor and a control circuit.

8. The end effector assembly of claim 1, wherein the first jaw member comprises a first opaque plug at a distal end of the first jaw member.

9. The end effector assembly of claim 8, wherein the first jaw member comprises a second opaque plug at a proximal end of the first jaw member.

10. The end effector assembly of claim 1, wherein the transparent tissue contacting surface or the transparent viewing portion or both comprises quartz or sapphire.

11. The end effector assembly of claim 1, wherein the second jaw member comprises a second transparent viewing portion.

12. The end effector assembly of claim 1, wherein the second jaw member comprises a second tissue contacting surface opposing the transparent tissue contacting surface of the first jaw member.

13. The end effector assembly of claim 12, wherein the second tissue contacting surface is transparent.

14. The end effector assembly of claim 1, wherein the first jaw member comprises one or more mounts configured to prevent the optical fiber from rotating within the fluid-tight cavity.

15. The end effector assembly of claim 1, comprising an optical feedback system.

16. The end effector assembly of claim 15, wherein the optical feedback system comprises an array of one or more photodiodes and a filter.

17. A surgical instrument comprising:
a handle;
an elongated body extending from the handle;
an optical fiber extending from the handle and through the elongated body; and
an end effector assembly secured to a distal portion of the elongated body, the end effector assembly comprising:
a first jaw member comprising:
a transparent tissue contacting surface;
at least one transparent viewing portion;
a fluid-tight cavity configured to receive an optical fiber; and
a reflector configured to reflect a substantial portion of light from the optical fiber towards the transparent tissue contacting surface; and
a second jaw member configured to move towards the transparent tissue contacting surface of the first jaw member.

18. The surgical instrument of claim 17, comprising a light source coupled to a proximal end of the optical fiber.

19. The surgical instrument of claim 17, wherein the end effector assembly is configured to cut and seal tissue with optical energy.

20. The surgical instrument of claim 19, wherein an output beam profile of the end effector assembly provides simultaneous cutting and sealing of tissue.

21. The surgical instrument of claim 17, comprising an optical feedback system.

22. The surgical instrument of claim 17, wherein the optical feedback system comprises an array of one or more photodiodes and a filter.

23. A method comprising:
providing light into an optical fiber of an end effector assembly, the end effector assembly comprising:
a first jaw member comprising:
a transparent tissue contacting surface;
at least one transparent viewing portion; and
a fluid-tight cavity receiving the optical fiber; and
a second jaw member; and
reflecting a substantial portion of light from the optical fiber towards the transparent tissue contacting surface of the first jaw member.

24. The method of claim 23, comprising moving a tip of the optical fiber within the fluid-tight cavity of the first jaw member and thereby cutting or sealing tissue or both.

25. The method of claim 23, comprising moving the second jaw member towards the transparent tissue contacting surface to clamp tissue between the first jaw member and the second jaw member.

26. The method of claim 24, wherein an output beam profile of the end effector assembly provides simultaneous cutting and sealing of tissue.

27. The method of claim 23, comprising controlling the light with an optical feedback system.

28. The method of claim 27, wherein the optical feedback system comprises and array of one or more photodiodes and a filter.
